(19) United States
(12) Patent Application Publication
(10) Pub. No.: US 2011/0295170 A1
(43) Pub. Date: Dec. 1, 2011

(54) ORTHOTIC DEVICE FOR THE CORRECTION OF DEFORMITIES OF THE VERTEBRAL COLUMN

(76) Inventors: Eusébio José Laranjeira Gomes, Porto (PT); Manuel Laranjeira Gomes, Porto (PT); Ana Mafalda Fontes Pinto Dos Reis, Porto (PT)

(21) Appl. No.: 13/127,690

(22) PCT Filed: Nov. 4, 2008
(86) PCT No.: PCT/PT2008/000046

§ 371 Date: Aug. 17, 2011

(51) Int. Cl. A61F 5/00

(52) U.S. Cl. 602/19

(57) ABSTRACT

An orthotic device can be intended for the correction of deformities of the vertebral column, in particular, of juvenile idiopathic scoliosis, and can be considered an external orthotic device or brace. The orthotic device can include four points of pressure: an axillary support; a lumbar support; a thoracic support, which, associated with the axillary support results in the opposite lateral inclination of the segment above the apical vertebra; and a hemi-hip, which can result in the ilium-trochanterian pressure and interacts with the supports of the lumbar pressure and of the thoracic pressure, causing the lateral inclination of the segment below the apical vertebra. At least one fixation bar can be provided to interconnect the different elements, allowing for the homogeneous distribution of forces and causing an oblique asymmetrical traction, and connecting elements allowing for the adjustment and fixing of the supports and bars.

This application is a U.S. national phase filing under 35 U.S.C. §371 of PCT Application No. PCT/PT2008/000046 filed Nov. 4, 2008, the entirety of which is hereby incorporated by reference herein.

The orthotic device of the disclosed subject matter is intended for the correction of deformities of the vertebral column caused by alterations in balance, making it thus applicable in the production of corrective prostheses.

### STATE OF THE ART

The orthopaedic treatment by means of a corrective brace constitutes the most effective and the least invasive approach in the therapy of juvenile idiopathic scoliosis (1-3).

The various imaging techniques, namely conventional radiology, take on an important role in the diagnosis and follow-up of this type of pathology (4). Technological developments in the field of imaging, most notably the development of software based on computerized tomography scans (CT) and magnetic resonance imaging (MRI) have resulted in an increase in the accuracy of diagnosis and facilitate the detection and/or confirmation of non-idiopathic aetiologies of scoliosis. New imaging techniques have been developed to obtain 3D images with minimum exposure of the patient to ionizing radiation, thus contributing to an accurate development of a brace that is closer to the physiological conditions of the organism in this type of pathology (5, 6).

The option of surgery is only taken into consideration in the most serious cases, between 10 and 15 percent (7-14).

In this text, no mention shall be made of older models of braces, in leather and steel, nor shall we discuss all the braces available in the market. Only those models of braces which are relevant to modern orthopaedic treatment, from the Milwaukee brace from the United States of America, will be considered.

The Milwaukee brace was conceived in 1957 by Blount and its constituent parts are: a hip with a fastening at the back, an anterior bar and two posterior bars supporting a cervical collar to which one mentonian support is added, two suboccipital supports and one thoracic support, in leather (15).

The main corrective quality of this brace is the self-elongation of the axis of the vertebral column achieved because the patient tries to avoid the occipital-mentonian supports. Although the pressure of the thoracic padding is not very important, since it leaves the thorax free, it does require close vigilance to avoid mandibular and dental deformities. This brace must be worn 23 hours a day for a period of years, the length of which is determined by the regression of the curvature. The need for prolonged use, together with its unattractive appearance and the discomfort it causes, results in lower compliance rates.

The disclosed subject matter differs from the Milwaukee brace in that it utilizes 4 pressure points on the frontal plane and an oblique and asymmetrical axillary and ilium-trochanterian traction force rather than the anteroposterior forces and the occipital-mentonian support, making it more comfortable and more aesthetically pleasing. The ilium-trochanterian pressure is supplied by a hemi-hip in a flexible material, in conjunction with a lumbar and thoracic support, thus inducing the lateral inclination of the segment below the apical vertebra. The result of the conjunction of the thoracic and the sub-axillary pressure points is an opposite lateral inclination of the segment above the apical vertebra. The traction force between the axillary and the ilium-trochanterian support maximizes the action of the 4 points of pressure.

The Lyonnais brace is traditionally made from a cast on the principle of EDF (elongation, derotation, and flexion). Its Plexiglas parts are mounted on a metallic frame. Its main drawbacks include not allowing for the regulation of the pressure forces and its excessive weight (16-18). The limited efficacy of the three pressure points, already proven, is overcome in our device by means of the 4^{th} pressure point and of the valves that allow for the regulation of the pressure applied.

The device here submitted presents the advantage of utilizing the 4 pressure points and a traction force, unlike the Lyonnais, which uses only 3 pressure points.

The Boston brace is produced from a thermo-mouldable prefabricated model with a posterior fastening. The principle on which it rests is that of lumbar delordosing in conjunction with derotation pads. It has little effect on the frontal plane, since it is based on the principle that scoliosis is caused by hyperlordosis and rotation. A univalve brace such as this one presents some drawbacks in terms of compliance rates, since it can be uncomfortable in hot weather conditions, and the fact that the correction is achieved solely through anteroposterior forces does not allow for a satisfactory degree of correction of the curvatures. It is only effective in lumbar and low dorsolumbar scoliosis, and it shows no efficacy in the treatment of dorsal scoliosis.

The disclosed subject matter here submitted privileges an integrated action of several pressure points distributed alongside the torso, and a traction force, increases its efficacy in the treatment of upper dorsal and dorsolumbar scoliosis.

The Cheneau brace is based on research on multiple pressure points (52, according to its inventor), but, nevertheless, it still privileges the three classical pressure points, one lumbar, one dorsal and one axillary. It resorts to hyperpressure as a means of obtaining hypercorrection. When tolerated, it can achieve positive results, but vigilance is recommended on the possibility that it may cause costal deformations.

In the disclosed subject matter here submitted, the four pressure points and the oblique and asymmetrical traction force simplify the corrective mechanism and result in a higher degree of efficacy.

With the braces meant exclusively for night-time wear, such as the Charleston and the Providence, satisfactory corrections are achieved, but their efficacy is significantly reduced due to the limited number of hours in which they are worn. Furthermore, these are univalve braces, with the drawbacks already mentioned, leading to lower compliance rates because of the lack of comfort and the restriction of movements associated to their use that render them unsuitable for day-time wear.

Recently, so-called dynamic braces have come on the market, such as the Spinecor. These are very comfortable and allow for a greater mobility, because they are constituted by adjustable cloth bands intended to induce a corrective posture. Nevertheless, no scientifically proven results have yet been shown.

The principle on which our device is based is the combination of an oblique asymmetrical traction force between the axillary and the ilium-trochanterian area, with four pressure points instead of the three pressure points used in the braces already mentioned. Our device allows for a better effect of elongation/traction and alignment of the vertebral axis. Summary

The constituent parts of the orthotic device are an axillary support (1), a lumbar support (3), a thoracic support (4), a hemi-hip (5) in a mouldable material and at least one fixation bar (2) which can be in a rigid material.

Four pressure points are used, combined with a sub-axillary and ilium-trochanterian traction force, inducing an opposite lateral inclination in the vertebral segments above and below the apical vertebra, which causes the translation in an opposite direction of the scapular and the pelvic girdles on the frontal plane.

The specificity of the pressure points results in a higher rate of compliance with wearing the brace.

The disclosed subject matter is thus applicable medically, since it seeks to correct scoliosis by placing the patient in a position of physiological rotation of the torso through the combination of four points of pressure with an axillary and an ilium-trochanterian traction from opposite sides, causing the translation in opposite directions of the scapular and the pelvic girdles on the frontal plane.

The exemplary orthotic device is based on the combination of an oblique and asymmetrical traction force enabled by the traction bar (2) interacting with the four pressure points in the axillary (1), lumbar (3), thoracic (4) and hemi-hip (5) supports. They decrease the frontal curvatures, causing the opposite lateral inclination of the vertebral segments above and below the apical vertebra, resulting in a translation in the opposite direction of the scapular and pelvic girdles on the frontal plane.

This device presents the advantage of an economy in materials used, since it is constituted by several small parts (supports and hemi-hip) connected by means of bars, unlike univalve braces (one part), and it is also more ergonomic, given its adaptation to the body of the patient.

### BRIEF DESCRIPTION OF THE FIGURES

FIG. 1: Schematic representation of the orthotic device for the correction of deformities of the vertebral column

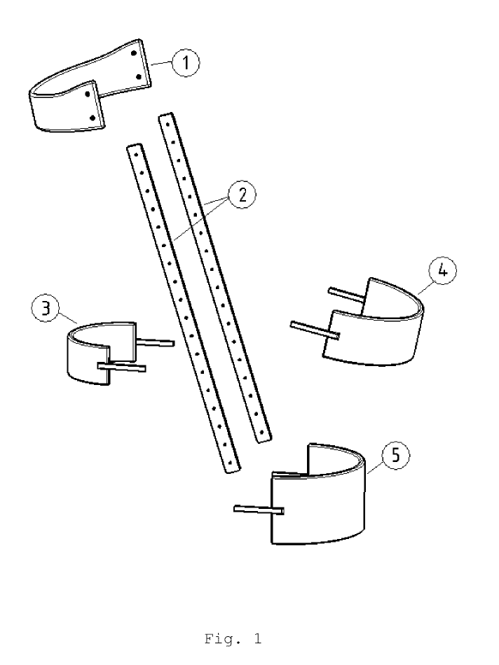

axillary support

fixation bar

lumbar support

thoracic support

hemi-hip

FIG. 2: Schematic representation of the application of the orthotic device for the correction of deformities of the vertebral column.

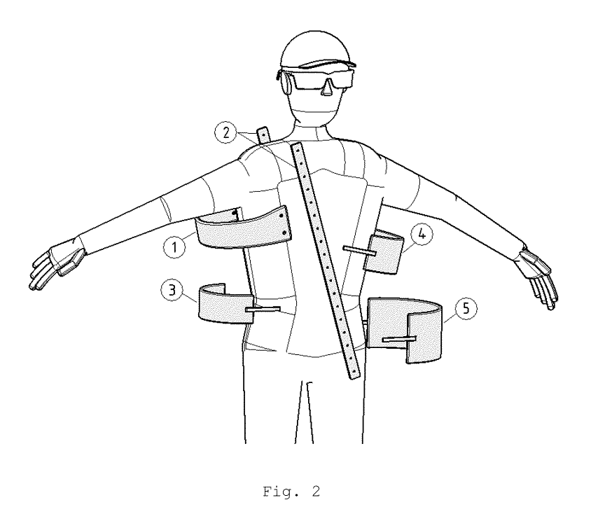

FIG. 3: Schematic representation of the pressures and force exerted.

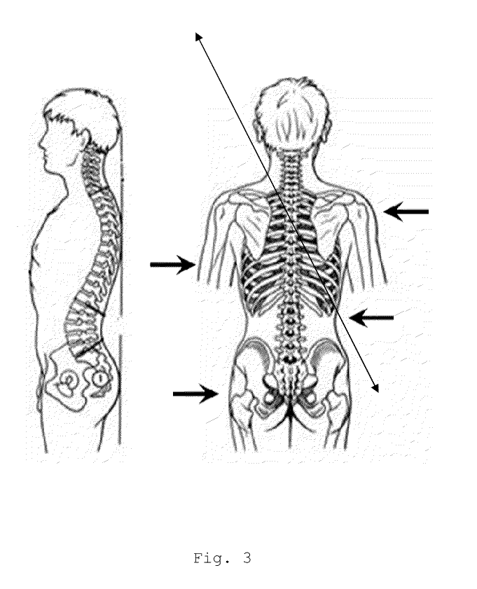

### GENERAL DESCRIPTION OF THE DISCLOSED SUBJECT MATTER

The orthotic device hereby submitted is intended for the correction of deformities of the vertebral column, namely idiopathic scoliosis. In one embodiment it can be constituted by four separate structures, or four points of pressure:

an axillary support (1);

a lumbar support (3);

a thoracic support (4), which, associated with the axillary support (1) results in the opposite lateral inclination of the segment above the apical vertebra;

a hemi-hip (5), which results in the ilium-trochanterian pressure and interacts with the supports of the lumbar pressure (3) and of the thoracic pressure (4), causing the lateral inclination of the segment below the apical vertebra.

and at least one fixation bar (2) to interconnect the different elements, allowing for the homogeneous distribution of forces and causing an oblique asymmetrical traction, and connecting elements allowing for the adjustment and fixing of the supports and bars.

The disclosed subject matter can include an axillary support (1), a lumbar support (3), a thoracic support (4) and a hemi-hip (5) in a mouldable material, possibly upholstered for comfort, and at least one fixating bar (2) in a rigid material.

In another version, the orthotic device should include two fixation bars (2), an anterior and a posterior one, to interconnect the different elements which distribute the forces homogeneously, thus achieving an oblique asymmetrical traction, and connecting parts allowing for the adjustment and fixing of said supports and bars.

The mouldable material of the supports may be a composite material, such as carbon fibre, polypropylene, polyethylene, “ortochoc” (a rigid and durable copolymer. It is a sheer material, with a yellow tint, and its moulding temperature is a maximum of 170° C.), amongst others.

The rigid material of the fixation bars may be an aluminium, steel or carbon fibre alloy, amongst others.

The connecting elements which enable the adjustment and fixing of the supports and bars may be bolts, screws, Velcro, buttons, or clasps, amongst others.

The upper contour of the axillary support (1) rests on the pectoral, grand dorsal and shoulder blade muscles and in the anterior part it should be subclavicular.

The thoracic support (4) should follow the exact contour of the rib corresponding to the apical vertebra.

The lumbar support (3) follows the contour above the iliac crest, avoiding the anterior third of the eleventh and twelfth ribs.

The hemi-hip (5) follows the contour of the anterior upper third of the iliac crest exerting pressure on the medium gluteus and on the greater trochanter.

The bars (2) are applied on fixed points in the anterior and posterior parts of the axillary support (1) and of the hemi-hip (5). The thoracic (4) and lumbar (3) supports are fixed by means of a flexible material.

The perfect adaptability of the components to the anatomy of the patient is essential to create a play of forces in which the sub-axillary and ilium-trochanterian opposite traction, combined with the lateral pressures, create a lateral antagonistic inclination in the vertebral segments above and below the apical vertebra, corresponding to the opposite translation of the scapular and pelvic waists on the frontal plane.

One point of departure for the presently disclosed subject matter was the observation that the rotation of the torso in the direction of the thoracic deviation in patients with idiopathic scoliosis resulted in the automatic correction of the curvature. It was also observed that on a thoracic level, there was an apparently paradoxical costal deformation. In fact, in the rotation of the torso to the right, the costal convexity appears on the left and its opposite occurs on the right.

In its turn, the anteroposterior diameter of the left costal cage increases and the right decreases, while the front diameter increases on the right and decreases on the left.

Based on this data, we developed an orthotic device to enable us to place the column in rotation. In practice, this would translate in a position of opposite rotation of the scapular and of the pelvic girdles. Several attempts at the practical application of this principle did not in the end achieve satisfactory results. We were thus compelled to carry out studies of the biomechanics and cinematic of the vertebral column, which enabled us to reach the conclusions that resulted in the present model of brace.

The rotation of the torso causes two curvatures in opposing directions, an upper cervicodorsal one and a lower dorsolumbar one. Contrary to our expectations, the pelvic and the scapular girdles rotate in the same direction and not in opposite directions. Thus, rotation does not cause a torsion of the vertebral column, but only the opposite lateral inclination of the vertebrae above and below D7-D8 with an opposite translation of the girdles on a frontal plane, maintaining its relative parallelism.

We also concluded that the axial rotation of the vertebral body is synonymous with torsion. Whenever the column presents with elements of rotation/torsion, three vertebral curvatures (scoliosis) are formed.

Based on these conclusions, we developed an orthotic device to induce the opposite inclination between the vertebral segments above and below the apical vertebra, resulting in a physiological rotation of the torso.

The aim of the posture thus obtained is to transform the three pathological curvatures that are typical of scoliosis in two physiological curvatures, which, depending on the deformation, translates into corrected curvatures.

The axillary support (1) should be adapted to the contour of the axilla and exert pressure on the shoulder blade muscles on the back and on the greater pectoral muscles on the front.

The thoracic support (4) must follow the exact contour of the rib corresponding to the apical vertebra.

The lumbar support (3) follows the contour above the iliac crest skirting the anterior third of the eleventh and of the twelfth ribs.

The hemi-hip follows the contour of the anterior upper third of the iliac crest and applies, exerting pressure on the medium gluteus and on the greater trochanter.

The bars (2) are applied on fixed points in the anterior and posterior parts of the axillary support (1) and of the hemi-hip (5). The fixing of the thoracic support (4) and of the lumbar support (3) is accomplished by means of a flexible material.

The conception of the device of the disclosed subject matter decreases the risks of costal deformations due to the specificities of the points of pressure. Furthermore, it improves tolerance to increases in temperature, since it does not cover the trunk because it is multivalve (constituted by several pieces).

Additionally, it is more comfortable to wear, since its principle of application of forces increases the tolerance to pressures.

The model of orthotic device developed is the result of intensive research leading to the conclusion that the principle of the multivalve device (constituted by several pieces) to correct the alteration of the distribution of forces in the organism, the cause of postural deviations on the frontal plane (scoliosis) is preferable.

1. Orthotic device comprising four points of pressure:
an axillary support;
a lumbar support;
a thoracic support, associated with the axillary support;
a hemi-hip, exerting ilium-trochanterian pressure and interacting with the supports of the lumbar pressure and of the thoracic pressure,
and at least one fixation bar to interconnect the different elements, wherein combining such points of pressure with a sub-axillary and ilium-trochanterian oblique asymmetrical traction force, inducing an opposite lateral inclination in the vertebral segments above and below the apical vertebra, which causes the translation in an opposite direction of the scapular and the pelvic girdles on the frontal plane and in that the axillary support, lumbar support, thoracic support and hemi-hip are made of a mouldable and temperature resistant material.

2. Orthotic device according to claim 1 further comprising two fixation bars, an anterior and a posterior one, which distribute the forces homogeneously.

3. Orthotic device according to claim 2 wherein the mouldable material is a composite, carbon fibre, polypropylene or polyethylene.

4. Orthotic device according to claim 1 wherein the fixation bars are made of a rigid material.

5. Orthotic device according to claim 4 wherein the rigid material is an aluminium, steel or carbon fibre alloy.

6. Orthotic device according to claim 1 wherein the fastening elements are bolts, screws, Velcro, buttons or clasps.
